(19) United States
(12) Patent Application Publication
(10) Pub. No.: US 2018/0303393 A1
(43) Pub. Date: Oct. 25, 2018

(54) DETERMINATION OF GASES DISSOLVED IN BLOOD IN THE EXTRACORPOREAL CIRCULATION

(71) Applicant: FRESENIUS MEDICAL CARE DEUTSCHLAND GMBH, Bad Homburg (DE)

(72) Inventor: Andreas MAIERHOFER, Schweinfurt (DE)

(73) Assignee: Fresenius Medical Care Deutschland GmbH, Bad Homburg (DE)

(21) Appl. No.: 15/763,265

(22) PCT Filed: Sep. 22, 2016
(86) PCT No.: PCT/EP2016/072548

§ 371 Date: Mar. 26, 2018

(30) Foreign Application Priority Data
Sep. 26, 2015 (DE) 10 2015 012 519.2

(51) Int. Cl. A61B 5/1455; A61M 1/36; G01N 33/49

(52) U.S. Cl. CPC A61B 5/14557; A61M 1/3609; A61B 5/14542; G01N 33/4925; A61M 1/3627

(57) ABSTRACT

The invention relates to a blood treatment device with an extracorporeal blood circulation, which uses a gas sensor to measure the concentration of a gas in the gas supernatant of the drip chamber of the extracorporeal blood circulation. The blood treatment device has an evaluation unit, which calculates the corresponding partial pressure of the gas in the blood from the values measured in the gas supernatant in the drip chamber.

### TECHNICAL FIELD

The invention relates to a blood purification device machine having an extracorporeal blood circulation, a gas sensor and an evaluation unit so that the partial pressure of a gas in the extracorporeal blood circulation can be determined and monitored, as well as a method for determining the partial pressure of a gas in blood.

### BACKGROUND

In performing blood purification treatments, which are associated with an extracorporeal blood circulation, monitoring of the patient's parameters, in particular the vital parameters, is extremely important.

In dialysis clinics, this is typically done through regular observation of the patient by the nursing personnel. However, in long-term dialysis overnight or in home dialysis, there is only limited monitoring by nursing personnel or none at all.

Additional possibilities occur through automated monitoring, which is performed automatically by the dialysis machine. Examples of this include blood pressure measurements, electrocardiograms and monitoring with pulse oximeters. For these measurements, however, it is necessary to use additional sensors, but that is often difficult in home dialysis and frequently also disturbs the patient's sleep.

If determination of the partial pressure of a gas in blood is used for monitoring a vital parameter, this is usually done by performing measurements in the patient's respiratory air, in which these substances enter the alveoli through gas exchange (“Potential Application of Exhaled Breath Monitoring in Renal Replacement Therapy,” Kelly et al., Poster Presentation ASN 2113).

Examples of blood gases that supply relevant information for monitoring of patients include carbon dioxide, acetone and ammonium.

Determination of acetone, for example, can supply helpful information in treatment of diabetes patients because this permits diagnosis of ketoacidosis. Recording the values over multiple treatments can be helpful in dosing insulin for patients during dialysis.

Determination of the ammonium concentration in the breath is associated with the blood urea concentration (“correlation of breath ammonia with blood urea nitrogen and creatinine during hemodialysis,” Marashimhan et al. PNAS, vol. 98, p. 4617ff). Therefore, practically all analyses that would otherwise be based on determination of the blood urea concentration can be performed by an ammonium sensor.

Monitoring of the CO₂ partial pressure of blood, which is directly related to respiration and/or oxygen saturation and is thus an important vital parameter is of particular interest and also the most widespread. An increase in CO₂ partial pressure may indicate inadequate respiration or even lack of respiration as in sleep apnea, for example. Sleep apnea affects mainly the elderly and the obese (in some cases also associated with other neurological disorders). The prevalence is estimated at 18 million patients in the United States. Dialysis patients are disproportionately affected because of their demographics and the co-morbidities that are often typical for them. With an ESRD prevalence of approximately 600 ppm and an assumed elevated sleep apnea prevalence by a factor of 2 in comparison with the normal population, in the United States alone >2000 dialysis patients are thus affected by sleep apnea. Sleep apnea results in a lower quality of sleep and can lead to cardiovascular diseases such as hypertension, right ventricular failure and even sudden cardiac death.

For example, the CO₂ content of the respiratory air is used in traditional apnea monitoring. To do so, the CO₂ content of respiratory air can be determined by analysis of the air flowing through a breathing mask or the air flowing through a tube inserted into the nose (end tidal CO₂ monitoring=ETCO₂ monitoring). Alternatively, determination of blood by transcutaneous CO₂ measurements or even blood gas analysis on the basis of blood samples can be used. All these methods require additional equipment, sensors attached to the patient and especially well-trained personnel. They are associated with burdens for the patient, such as dialysis patients, ranging from restricted comfort due to the attachment of additional sensors and possible skin irritation due to heating of the sensor in transcutaneous CO₂ monitoring to blood samples taken for blood gas analysis. Noninvasive methods of CO₂ monitoring are also subject to error. Mouth breathing instead of nose breathing can distort the measurement result in ETCO₂ monitoring in particular, but inadequate circulation in the area of skin beneath the sensor can lead to faulty results in transcutaneous CO₂ monitoring.

Another application of the CO₂ concentration determination specifically in dialysis patients is monitoring of the pH status of blood to detect acidosis.

WO 2013/156435 thus describes a device for extracorporeal blood treatment in which the CO₂ partial pressure is determined directly by measuring the bicarbonate concentration and the blood pH. The data for adjusting blood pH by regulating the bicarbonate concentration in the dialysate as a function of the resulting measurement data is used here. This requires the sensor to come in direct contact with the patient's blood, which entails the risk of cross contamination.

The object of the present invention is therefore to ensure safe automatic and continuous monitoring of a patient parameter, in particular the CO₂ partial pressure of blood, without requiring additional materials for disposable materials or incurring additional burdens for the patient.

### SUMMARY OF THE INVENTION

This object is achieved by a blood treatment device according to Claim 1 and the method according to Claim 15. Special embodiments of the invention are the subject matter of the dependent claims.

In one embodiment, a blood treatment device with an extracorporeal blood circulation has the standard components of a blood tubing system. This blood tubing system comprises a first line, which can be connected to the patient at a first end for withdrawal of blood and is connected to the compensating chamber at the second end. A first blood pump, which pumps the blood withdrawn from the patient into the compensating chamber in the extracorporeal circulation, is provided in this first line. Then a blood level develops in this compensating chamber so that the blood comes in contact with a gas space located above the blood level. From the compensating chamber, the blood is sent through a second line to the blood purification unit. In addition, the blood treatment device according to the invention has at least one gas sensor that can be connected to the compensating chamber for measuring the concentration of a gas in the gas space of the compensating chamber. It may be assumed that the equilibrium concentrations established in the gas chamber above the blood level in a compensating chamber of an extracorporeal circulation is similar to those in the pulmonary alveoli. In addition, the blood treatment device also includes an evaluation unit for readout and evaluation of the data measured by the gas sensor.

Positioning of the gas sensor in such a way as to permit noncontact, largely maintenance-free measurement that is also safe from cross contamination is advantageous. It is also advantageous that the consumable materials used as the standard in the blood treatment can also be used for the extracorporeal tubing system.

A sensor for carbon dioxide or ammonium or acetone may be used as the gas sensor, depending on the parameters and/or the parameters that should be monitored. It is also conceivable to combine various gas sensors.

Gas sensors for carbon dioxide, for example, for the relevant concentration range, are available commercially and are based on a determination of the specific adsorption of radiation in the infrared range, for example. These sensors can be used up to 100% relative humidity under non-condensable conditions.

To prevent condensation on the gas sensors, they may be designed to be heatable.

In analysis of the measured data, taking into account the blood temperature leads to a more accurate result, so a temperature sensor may be provided in the extracorporeal circulation, for example, in the inlet line, the outlet line or in the compensating chamber, supplying measurement results to the evaluation unit, which takes them into account in evaluating the measured data from the gas sensor.

Another parameter that can lead to an improvement in the accuracy of the measurement result in analysis of the measured data is the pressure prevailing currently on the gas sensor. An embodiment of the blood treatment device according to the invention may have a pressure sensor, which can measure the pressure in the gas space in which the gas sensor is arranged and which supplies the measurement results to the evaluation unit that takes them into consideration in evaluating the measured data from the gas sensor.

Whereas the compensating chamber is advantageously part of the blood tubing system, the gas sensor and the evaluation unit may be integrated into the blood treatment device. In an alternative embodiment, the gas sensor and the evaluation unit may be embodied as a separate module which can be connected to the blood treatment device.

The connection of the compensating chamber to the gas sensor may be provided by a connecting line, in particular a connecting tubing. A hydrophobic filter may be provided between the connecting tubing and the gas sensor to prevent contamination of the blood treatment device by blood.

Transport due to simple diffusion may be too slow, depending on the length of the connecting line, so the gas concentration established in the compensating chamber directly above the blood level must be actively transported to the gas sensor.

To do so, the blood treatment device according to the invention may have means for generating a gas flow. These means can induce a lift in the blood level in the compensating chamber by means of a flow restriction in the second line of the extracorporeal blood circulation and thus cause a shift in the gas volume out of the compensating chamber, or alternatively, these means may be provided as direct gas conveyance means.

To shift the blood level in the compensating chamber, a valve may be provided in the second line in the extracorporeal circulation. This valve can be controlled by the control and evaluation unit, so that it establishes a lower blood flow at certain points in time in the second line of the extracorporeal circulation than in the first line, so that the blood level rises in the compensating chamber, i.e., a rise in blood level is created. With the increase in blood level, a gas volume is displaced out of the compensating chamber to that gas sensor. To be sure that the concentration established in the compensating chamber also reaches the gas sensor, the displaced volume should preferably be at least twice as great as the volume in the connection between the compensating chamber and the gas sensor. With the tubing systems normally used in blood purification processes, the gas volume can be transported out of the compensating chamber and to the gas sensor with an increase in the blood level in the compensating chamber with a lift of approximately 1-2 cm.

Alternatively, a displacement of the level status can be accomplished by a second blood pump in the second line, which is activated by the control unit, so that it runs at a delivery rate, which is different from that of the first blood pump located upstream from the compensating chamber in the first line of the extracorporeal circulation. If the delivery rate of the second blood pump is lower than the delivery rate of the first blood pump, then there is an increase in the blood level in the compensating chamber when the second blood pump runs at a higher delivery rate than the first blood pump.

To prevent an increase in pressure on the gas sensor or the compensating chamber with an increase in level, the blood treatment device according to the invention may have compensating means, more specifically pressure compensating means arranged downstream from the gas sensor. These compensating means may be valves which open to the environment or to a compliance vessel. A pressure compensation is achieved in the gas sensor due to this opening.

The blood treatment device according to the invention may also have a compressor, which can cause a reduction in the blood level by introducing ambient air, which can thus restore the blood level in the compensating chamber.

For a renewed measurement of the gas concentration in the compensating chamber, the blood level may be then raised again.

Preferably intermittent measurements of the gas concentration are possible with this arrangement. The intervals of time may be selected to be short enough so that close screening of the concentration profile is possible.

Alternatively, the blood treatment device according to the invention may have a gas conveyance means, e.g., miniaturized bellows, which actively create a continuous gas flow. The compensating chamber is therefore designed so that, in addition to the first connecting tubing, it is also connected to the gas sensor by additional connecting tubing as a return tubing. The control unit of the blood treatment device is designed so that the gas conveyance means is to be controlled so that a circulating gas stream is created and the gas concentration can be measured continuously.

An ideal range for the measured gas concentration or the partial pressure of the gas in the blood can be stored in the evaluation unit, which calculates the partial pressure of the gas in the blood from the measured data from the gas sensor. If values outside of this target range occur when measuring the gas concentration, then a signal can be output by the evaluation unit.

This signal may be relayed to an alarm device, for example, which outputs an alarm signal so that additional measures are triggered.

In addition, the measured data can be used in controlling the dialysate composition, for example, in adjusting the bicarbonate concentration in the dialysis solution in the case of a dialysis treatment.

The blood treatment device according to the present teaching may operate according to the principle of hemodialysis, hemofiltration or hemodiafiltration or plasmapheresis. The blood treatment unit that is used may thus be designed as a dialysis filter or hemofilter or plasma filter.

A method according to the present teaching determines the partial pressure of a gas in blood in an extracorporeal blood circulation in a blood treatment device according to claim 1 by measuring the gas concentration in the compensating chamber by means of a gas sensor and calculating the partial pressure in blood, e.g., the p_{CO2} in blood, by analyzing the measured data of the gas sensor by means of the control and evaluation unit.

To bridge a spatial distance between the compensating chamber and the gas sensor, it may be provided that a gas flow is produced from the compensating chamber to the gas sensor.

The gas flow can be established by raising the blood level in the compensating chamber. raising blood level in the compensating chamber causes a displacement of the gas in the compensating chamber into the connecting line to the gas sensor. To be certain that the concentration established in the compensating chamber reaches the gas sensor, the displaced volume should preferably be at least twice as great as the volume in the connecting tubing. With the conventional commercial blood tubing systems, a lift of 1-2 cm in the compensating chamber may be sufficient to transport the gas in the compensating chamber to the gas sensor. This lift can be achieved by an intermittent flow restriction in the blood circulation downstream from the compensating chamber. To do so, the evaluation and control unit may trigger a valve located in the second line, so that it closes partially or even completely for a short period of time. At the same time, corresponding means such as a throttle valve or a valve to a compliance container may be opened for compensating the pressure, so that there is no excess pressure on the gas sensor. A subsequent reduction in the level can then be reversed by a pressure surge through a compressor.

Alternatively, a shift in the level in the compensating chamber can be achieved by means of a second blood pump in the second line, the drain line. Different delivery rates can be established for the first and second blood pumps by triggering the evaluation and control unit. If the blood pump delivers blood at a higher delivery rate than the second blood pump, there is an increase in the blood level in the compensating chamber. Conversely, a delivery rate of the first blood pump, which is lower than that of the second blood pump, results in a reduction in the blood level in the compensating chamber.

However, the gas flow can also be accomplished by an active transport of the gas through a gas delivery means.

### BRIEF DESCRIPTION OF THE DRAWINGS

FIG. 1: Schematic diagram of a first embodiment of the blood treatment device according to the invention.

FIG. 2: Schematic diagram of a second embodiment of the blood treatment device according to the invention.

FIG. 3: Correlation between predialytic p_{CO2} and bicarbonate.

FIG. 4: Simulated p_{CO2} curve during long-term dialysis with apnea.

### DETAILED DESCRIPTION OF AN EXEMPLARY EMBODIMENT

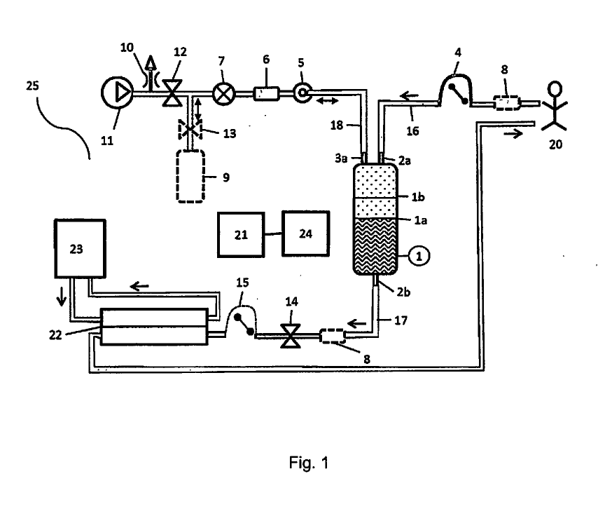

FIG. 1 shows a first embodiment of a blood treatment device (25) according to the present teaching. Blood is conveyed from the patient (20) by the blood pump (4) through the first line (16), the arterial line, and goes through the connection (2a) into the compensating chamber (1). A blood level (1a) develops in the compensating chamber (1). Connection (2a) may be situated above or below the blood level (1a), i.e., even adjacent to the connection (2b), which forms an outlet out of the compensating chamber. The blood then flows further out of the outlet (2b) of the compensating chamber (1), through the second line (17), to the blood purification unit (22), e.g., a dialyzer, and then back to the patient. The blood purification unit (22) is also connected to a dialysate preparation unit (23), where the dialysis solution can be prepared with the desired bicarbonate concentration.

Above the blood level in the compensating chamber (1), gases dissolved in blood are in equilibrium with a corresponding gas concentration in the gas chamber of the compensating chamber (1). The gas chamber in the compensating chamber (1) is connected to the gas sensor (6) by means of the connecting tubing (18). The gas sensor is arranged in the fixed part of the blood purification device, for example, the dialysis machine. To protect the gas sensor from contamination by blood, which might be able to rise into the connecting tubing in the event of a malfunction, a hydrophobic filter (5) is provided at the end of the connecting tubing (18). Downstream from the hydrophobic filter, the gas mixture is sent to a CO₂ sensor (6) optimized for a concentration measurement range of approximately 2-10 vol % CO₂, corresponding to the physiologically relevant CO₂ partial pressures of 15-80 mm Hg.

The measured data recorded from the CO₂ sensor is read out by the control and evaluation unit (21), and the CO₂ partial pressure is calculated on the basis of the formulas from the control and evaluation unit given below.

The relationship between the CO₂ concentration C_{CO2} in blood and the CO₂ partial pressure p_{CO2} in the gas space is described by Henry's law.

c_{CO}_{₂}=K_{H,CO}_{₁}p_{CO}_{₂} Equation 1

Henry's constant for CO₂ is a function of the temperature:

$${K}_{H , {CO}_{2}} ( T ) = {K}_{H , {CO}_{2}}^{x} exp \{ - \frac{{Δ}_{s} H}{R} ( \frac{1}{T} - \frac{1}{{T}^{x}} ) \} where {K}^{x} = 3.4 \cdot {10}^{- 4} \frac{mol}{{m}^{3} Pa} , {T}^{x} = 298.15 K and \\ \frac{{Δ}_{s} H}{R} = 2400 K . \qquad Equation 2$$

In addition, the CO₂ concentration C_{CO2} in blood is associated with the bicarbonate concentration c_{HCO3} according to the Henderson-Hasselbalch equation:

$${CO}_{2 , aq} + {H}_{2} O ↔ {H}^{+} + {HCO}_{3}^{-} \\ pH = {pK}_{s} + log \frac{{c}_{{HCO}_{3}^{-}}}{{c}_{{CO}_{2}}} \\ {c}_{{CO}_{2}} = {c}_{{HCO}_{3}^{-}} {10}^{- ( pH - {pK}_{s} )} \\ {p}_{{CO}_{2}} ( T ) = \frac{1}{{K}_{H , {CO}_{2}} ( T )} {c}_{{HCO}_{3}^{-}} {10}^{- ( pH - {pK}_{s} )} where \\ pKs \approx 6.1 \qquad Equation 3$$

The CO₂ partial pressure p_{CO2} established in the gas space above the blood level corresponds to the CO₂ partial pressure measured by the blood gas analyzer at the same temperature.

The temperature prevailing at a given time is determined by the temperature sensor (8) arranged in the line (17).

Like the temperature, the normal pressure must also be taken into account for an accurate determination of the partial pressure. In compression of the gas mixture to a pressure p not equal to normal pressure pₙₒᵣₘ, the CO₂ partial pressure under normal conditions is obtained as

$${p}_{{CO}_{2}}^{norm} = {p}_{{CO}_{2}} ( {p}_{tot} ) \frac{{p}_{norm}}{{p}_{tot}} \qquad Equation 4$$

The temperature- and pressure-compensated CO₂ partial pressure (p_{CO2}(T, pₙₒᵣₘ)) in the blood is then calculated from the CO₂ concentration in the gas space above the blood level with the help of equations 1 and 4.

$${p}_{{CO}_{2}} ( T , {p}_{norm} ) = \frac{1}{{K}_{H , {CO}_{2}} ( T )} \frac{{p}_{norm}}{{p}_{tot}} {c}_{{CO}_{2}} \qquad Equation 5$$

Use of the conventional disposable materials in hemodialysis for the extracorporeal tubing system is especially advantageous here. The gas sensor is preferably arranged in the blood treatment device to permit a noncontact measurement that is reliable, largely maintenance-free and protected from cross-contamination.

The arrangement of the gas sensor in the blood treatment device and not directly in the compensating chamber also entails the advantage that no changes are necessary in the tubing systems used as a standard. The gas sensor is part of the hardware of the blood treatment device. Depending on the distance of the compensating chamber from the gas sensor, which is derived to a definitive extent from the length of the tubing connection (e.g., 10-30 cm), active transport of the gas concentration established over the blood level to the gas sensor is necessary.

Without auxiliary means, which create a flow from the gas space directly over the blood level to the measurement sensor, the transport of the gas concentration located at the blood/air contact point, in particular the carbon dioxide concentration, to the gas sensor is determined only by the diffusion. For one-dimensional diffusion of a gas into a space, which did not previously contain the gas to be considered, the maximum concentration of the diffusion front is as follows:

xₘₐₓ²=2Dtₘₐₓ

With a typical diffusion constant D˜1.6*10⁻⁵ m²/s, the gas requires several minutes for diffusion of several centimeters (e.g., xₘₐₓ=20 cm more than 20 min).

In the embodiment according to the invention shown in FIG. 1, various alternative means, a valve (14) and/or a second blood pump (15) (shown with dotted lines) are provided for the purpose of gas transport to the gas sensor (6) in the second line (17).

The two means (14, 15) to be used alternatively can be controlled by the evaluation and control unit (21), so that they create a flow restriction downstream from the compensating chamber (1). In the case of the valve (14), this flow restriction is created by closing the valve. In the case of the second blood pump (15), the flow restriction due to the setting of a delivery rate of the second pump (15) which is lower than the delivery rate of the first blood pump (4) becomes . . . [sic]. Due to the creation of a flow restriction in the second line (17), the flow level in the compensating chamber (1) is raised from position (1a) to (1b). Therefore, gas is displaced out of the gas space of the compensating chamber (1) and thus a flow through the gas sensor (6) is achieved. To be sure that the gas concentration established in the compensating chamber (1) reaches the gas sensor (6), the displaced volume should preferably be at least twice as great as the volume in the link between the connection (3a) of the connecting line (18) to the compensating chamber (1) and the gas sensor (6). With the blood tubing systems conventionally used in hemodialysis, a lift of approximately 1-2 cm is sufficient for this.

The air pressure built up in raising the blood level may either be reduced by opening the outlet valve (12) and allowing air to escape through the throttle valve (10) to the environment or, as an alternative, pressure compensation is made possible in a compliance vessel (9) (shown with dotted lines) after opening the compliance valve (13). In both alternative possibilities, an air flow is induced through the gas sensor (6), so that the gas concentration prevailing in the gas space of the compensating chamber (1) can then be measured on this gas sensor.

To restore the blood level (1b) to (1a), the valve (14) is opened again, and/or the second blood pump (15) is operated at a higher delivery rate. A pressure compensation can then be achieved by intake of ambient air by means of a compressor (11) and/or by means of the compliance vessel (9).

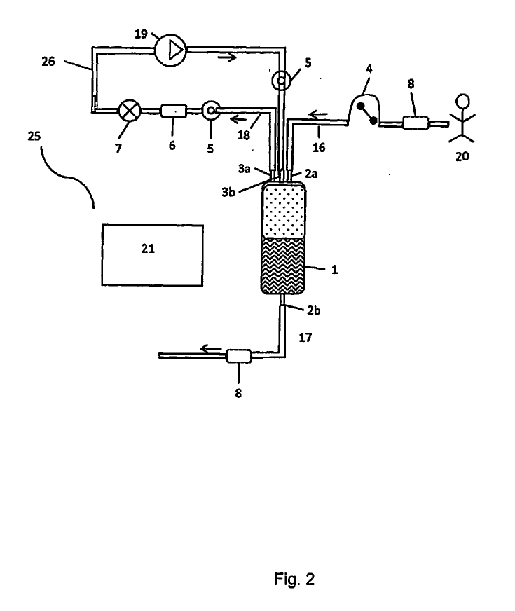

Alternatively, when using a compensating chamber (1) such as that shown in FIG. 2, a continuous flow through the gas sensor can be achieved. In this case, circulation from the connection (3a) by way of the gas sensor (6) by way of a return line (26) back to the connection (3b) can be achieved by means of a gas conveyance means (19) through the gas space of the compensating chamber (1). Hydrophobic filters (5) are provided here in the connecting line (18) and the return line as protection against contamination. The gas conveyance means (19) may be any type of means that would generate a low continuous air flow. For example, miniaturized balloon bellows, in which the movement of the legs is accomplished by means of piezoelectric elements, may be used.

The measured values picked up by the gas sensor (6) are read out by the evaluation unit (21), and the partial pressure of carbon dioxide in blood is determined using equation (5).

This partial pressure may provide indications of different pathological states.

Determining the Predialytic p_{CO2}

Values for the partial pressure of carbon dioxide measured at the start of the blood treatment differ only slightly from the predialytic partial pressure. A low value, in particular below the normal range of 35-45 mmHg usually is the cause of a metabolic acidosis with respiratory compensation. Thus, when a p_{CO2} below a defined threshold value is measured, a warning is output to the user by the dialysis machine (user interface/alarm unit (24)). This threshold value may be an absolute value (e.g., clinical normal range) or a value determined individually for this patient and stored in a memory unit, determined from the mean value of the past measurements for this patient, for example.

Correcting Metabolic Acidosis

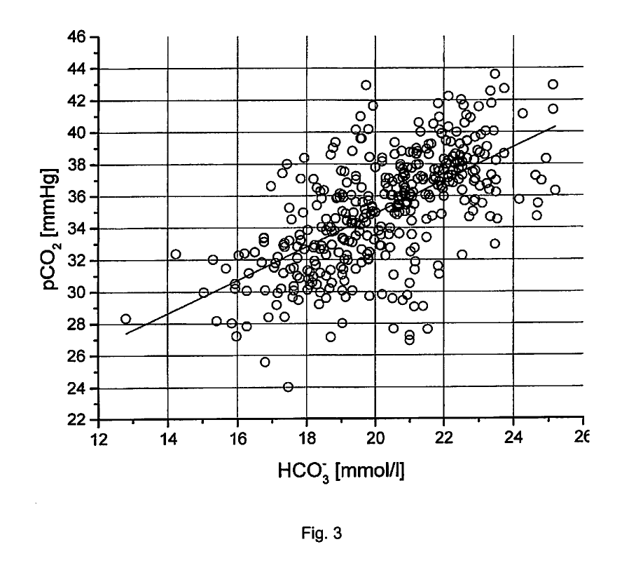

When metabolic acidosis occurs during dialysis treatment, it can be corrected by adding bicarbonate through the dialysis solution. FIG. 3 shows the correlation between predialytic p_{CO2} and the bicarbonate concentration HCO₃⁻ in the blood. After measuring the pCO₂ level, the bicarbonate concentration in the dialysate c_{D} can be adjusted by the dialysate preparation unit (23) of the dialysis machine in an automated process. To this end, a 1-pool model can be used, in which the distribution volume V of the patient, the dialysis dose Kt/V determined by means of online clearance measurements and the blood bicarbonate concentration c_{B,O} estimated from the pCO₂ measurement can be used.

$${c}_{B} ( t ) = {c}_{D} + ( {c}_{B , 0} - {c}_{D} ) {e}^{- α \frac{Kt}{V}} \qquad Equation 6$$

The factor α takes into account the lower clearance of bicarbonate in comparison with urea and is −0.7.

C_{B,0}=f(p_{CO}_{₂})=a₀+a₁p_{CO}_{₂} Equation 7

The coefficients a₀ and a₁ can be determined by a linear adjustment of the value pairs shown in FIG. 3. Since the metabolic processes depend on the patient, a better correspondence between the estimated level and the actual level can be achieved if the p_{CO2} and bicarbonate are determined over a certain period of time by means of a reference instrument (blood gas analyzer), and the coefficients a₀ and a₁ are determined for each individual patient.

If the Kt/V achieved at time t₁ can be estimated on the basis of the dialysis settings (blood, dialysate and substitute flows, dialysis characteristics), then the dialysate bicarbonate to be adjusted to achieve a blood bicarbonate concentration at time t₁ can be calculated from equation 8:

$${c}_{D} = \frac{{c}_{B} ( {t}_{1} ) - {c}_{B , 0} {e}^{- α \frac{Kt}{V}}}{1 - {e}^{- α \frac{Kt}{V}}} \qquad Equation 8$$

By repeated measurement of the p_{CO2} and the dialysis dose achieved so far, it is also possible to correct the adjustment of the dialysate bicarbonate during the dialysis.

Apnea Monitoring

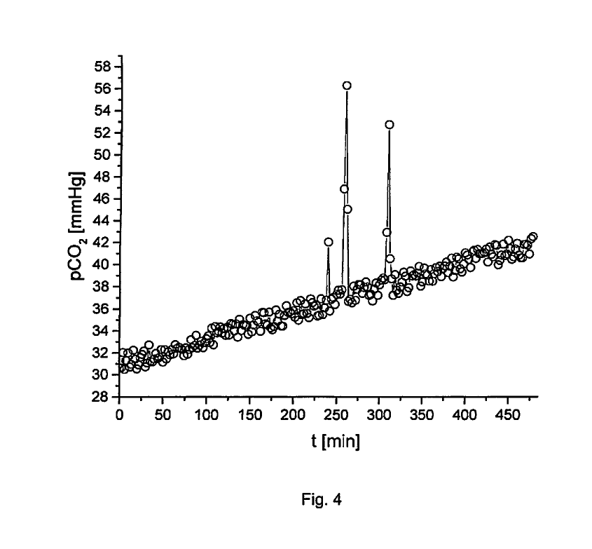

As already described in the introduction, faulty respiration may occur especially in long-term nocturnal dialysis because of errors in the physiological regulatory mechanisms or due to partial obstruction of the respiratory tract. This leads to an increase in the p_{CO2}. Studies on patients with obstructive sleep apnea have shown that the p_{CO2} is >11 mmHg higher in these patients when they wake up in the morning than before going to sleep (“Changes in the Arterial CO₂ During a Single Night's Sleep in Patients with Obstructive Sleep Apnea,” Chin et al., Internal Medicine, vol. 36, pp. 454ff). In transient respiratory arrest, which may last for one minute, it is to be expected that the rise in p_{CO2} will turn out to be much higher, as shown by the simulated p_{CO2} curve in FIG. 4 during long-term dialysis with apnea. These critical interruptions in respiration can thus be detected by means of the continuous monitoring of p_{CO2} described here.

Apnea can be diagnosed in a recording of the p_{CO2} curve and taken into account when planning therapy.

However, online monitoring to prevent longer periods of apnea is also conceivable. Various warning criteria may thus be stored in the blood treatment device (25) and/or in the evaluation unit (21), for example, exceeding an absolute concentration threshold, an increase in the p_{CO2} above a concentration threshold, which is calculated from the previous course, or the typical course of previous treatments. Other criteria, for example, a certain duration of the threshold being exceeded or a change in the chronological course of the p_{CO2}, e.g., a greatly accelerated increase in comparison with the previous course, may also be taken into account.

If these states, which are to be evaluated as critical, occur, then an acoustic signal may be generated, alarming the patient or the nursing personnel, for example.

It would also be conceivable to wake the patient or stimulate respiration by triggering a weak electrical impulse on the patient.

When using a breathing mask, an increase in the respiratory pressure and/or the air flow may occur.

Diabetes Monitoring

The course of metabolic diseases, e.g., ketoacidosis in diabetes, can be diagnosed by using an acetone sensor (Massick et al., Proc. SPIE 6386, Optical Methods in the Life Sciences, 638600 (Oct. 17, 2006)) and the course can be observed by recording the values over several treatments. This may be helpful in dosing insulin for diabetics on dialysis.

Determining the Efficiency of Dialysis

There is a correlation between the urea concentration and the ammonium concentration in the breath (“Correlation of breath ammonia with blood urea nitrogen and creatinine during hemodialysis,” Narasimhan et al., PNAS vol. 98, pp. 4617ff). Therefore, practically all analyses otherwise based on determination of the urea concentration in blood are possible by means of an ammonium sensor (e.g., “High sensitivity ammonia sensor using a hierarchical polyaniline/poly(ethylene-co-glycidyl methacrylate) nanofibrous composite membrane,” Chen et al., ACS Appl Mater Interfaces, vol. 24, pp. 6473ff) therefore the protein conversion (PCR=protein catabolic rate) can be deduced from the curve of the ammonium concentration at the start of successive dialyses. The dialysis efficiency Kt/V can be calculated from the intradialytic change in the ammonium concentration:

$$\frac{Kt}{V} = - ln \frac{ {c}_{{NH}_{3}} ( t )}{{c}_{{NH}_{3}} ( 0 )} \qquad Equation 9$$

In contrast with measurements on the dialysate side, no correction of the measured values with respect to flow rates is necessary here (cf. BBraun: Adimea).

Function Test on the Hydrophobic Filter

In one embodiment of the invention, the pressure sensor (7) may also be provided in addition to the gas sensor (6) for monitoring the pressure in the blood tubing system in the blood treatment device (25), so that the connecting tubing (18) leads to the gas sensor (6) and also to the pressure sensor (7). The measured data of the gas sensor (6) can then also be used for performing a function test on the hydrophobic filter (5). When using a device as shown in FIG. 1, in which pressure compensation takes place with respect to the ambient atmosphere, the permeability of the hydrophobic filter (5) can be tested by means of the gas sensor (6) and the evaluation unit (21). Due to wetting of the hydrophobic filter (5) with blood or dialysis fluid, it also becomes impermeable for gas, so that a reliable pressure measurement is no longer possible. Since the CO₂ concentration established on the basis of the blood p_{CO2} in the gas space of (1) amounts to approximately 5%, but the normal CO₂ content of the ambient air is only approximately 400 ppm=0.004%, it is possible, when raising and lowering the level in the compensating chamber (1), to ascertain whether there is a gas connection between the compensating chamber (1) and the gas sensor (6). In the case of a tubing system filled with blood, an increase in CO₂ concentration to >1% is to be expected when raising the level, and a drop to less than 0.1% is to be expected when lowering the level. If this does not occur, then it is possible to conclude that the hydrophobic filter (5) is blocked.

The embodiments described here offer the possibility of determining and monitoring the concentration of gases dissolved in blood during an extracorporeal blood treatment without using additional sensors or blood sampling and analysis. The measurement of the gas concentration may take place using the conventional blood tubing systems, in a contact-free operation without contamination, so there are no additional stresses or risks for the patient. The measured values can be used for determining the efficiency of the treatment or as a function test for components of the extracorporeal circulation.

### LIST OF REFERENCE NUMERALS

compensating chamber (1)
liquid level (1a)
liquid level (1b)
connection chamber (2a), (2b)
connection chamber (3a)
blood pump (4)
hydrophobic filter (5)
gas sensor (6)
pressure sensor (7)
temperature sensor (8)
compliance vessel (9)
throttle valve (10)
compressor (11)
outlet valve (12)
compliance valve (13)
valve (14)
blood pump (15)
arterial portion of blood tubing system (16)
venous portion of blood tubing system (17)
connecting tubing (18)
gas conveyance means (19)
patient (20)
evaluation unit (21)
blood purification unit (22)
dialysate preparation unit (23)
user interface/alarm unit (24)
blood treatment device (25)
return line (26)

1. A blood treatment device (25) having an extracorporeal blood circulation comprising a first line (16) that can be connected to a patient (20) at one end for withdrawal of blood and connected to a compensating chamber (1) at the other end, a first blood pump (4) for conveying blood in the extracorporeal circulation into the compensating chamber (1), so that a blood level (1a, 1b) develops therein and contact of the blood with a gas space above the blood level is formed, and a second line (17) for conveying the blood further from the compensating chamber (1) to a blood purification unit (22), characterized in that the blood treatment device (25) comprises a gas sensor (6) that can be connected to the compensating chamber (1) for measuring the concentration of a gas in the gas space and an evaluation unit (21) for readout and evaluation of the measured data of the gas sensor (6).

2. The blood treatment device (25) according to claim 1, characterized in that the gas sensor (6) is a sensor for carbon dioxide or ammonium or acetone.

3. The blood treatment device (25) according to claim 1, characterized in that the gas sensor (6) can be heated.

4. The blood treatment device (25) according to claim 1, characterized in that a temperature sensor (8) for determining a measured value of the blood temperature is arranged in the first line (16), the second line (17) or the compensating chamber (1) of the extracorporeal blood circulation, wherein the evaluation unit (21) is designed to take into account the measured value for temperature compensation.

5. The blood treatment device (25) according to claim 1, characterized in that it has a pressure sensor (7) for determining a measured value for the pressure in the compensating chamber (1), wherein the evaluation unit (21) is designed to take into account the measured value for pressure compensation.

6. The blood treatment device (25) according to claim 1, characterized in that the compensating chamber (1) is connected by means of at least one connecting line (18), preferably a connecting tubing (18), to the gas sensor (6).

7. The blood treatment device (25), characterized in that a hydrophobic filter (5) is arranged in the connecting line (18) between the gas sensor (6) and the compensating chamber (1).

8. The blood treatment device (25) according to claim 1, characterized in that it has means for creating a gas flow out of the compensating chamber (1) through the connecting line (18) to the gas sensor (6), and the evaluation unit is designed as a control and evaluation unit (21).

9. The blood treatment device (25) according to claim 8, characterized in that the means for generating the gas flow are designed as a valve (14) or as a second blood pump (15) in the second line (17) of the extracorporeal blood circulation, wherein the valve (14) or the second blood pump (15) is controlled by the control and evaluation unit (21) so that a lower blood flow rate occasionally prevails in the second line (17) than in the first line (16) upstream from the compensating chamber (1), so that a displacement of the blood level (1a) in the compensating chamber (1) can be created.

10. The blood treatment device (25) according to claim 9, characterized in that it has compensating means (9, 10, 11 12, 13) for an increase in pressure occurring with a shift in blood level.

11. The blood treatment device (25) according to claim 8, characterized in that the gas sensor (6) and the compensating chamber (1) are additionally connected to a return line (26), wherein a gas conveyance means (19) is arranged in this return line (26), so that a circulating gas stream develops between the gas sensor (6) and the compensating chamber (1).

12. The blood treatment device (25) according to claim 1, characterized in that the blood purification unit (22) is a dialysis filter or a hemofilter or a plasma filter.

13. The blood treatment device (25) according to claim 1, characterized in that the evaluation unit (21) is designed to determine the partial pressure of a gas in the blood, and the target range for the partial pressure of the gas is saved in the evaluation unit (21), and in the case of a measured partial pressure outside of the ideal range, a signal is forwarded to an alarm device (24) which then outputs an alarm signal.

14. The blood treatment device (25) according to claim 1, characterized in that it has a dialysis preparation unit (23) for supplying the blood purification unit (22) with dialysate, and the evaluation unit (21) is designed as an evaluation and control unit to determine a partial pressure of the gas from the measured values of the gas sensor (6) and to assign a composition of the dialysate to the partial pressure of the gas, which is then supplied by the dialysate preparation unit (23).

15. A method for determining the partial pressure of a gas in blood of the extracorporeal blood circulation of a blood treatment device (25) according to claim 1, characterized in that the concentration of gas is measured by means of a gas sensor (6) in the gas space of the compensating chamber (1) and the partial pressure of the gas in blood is calculated from these measured data by the evaluation unit (21).

16. The method for determining the partial pressure of a gas in blood according to claim 14, characterized in that a gas flow from the compensating chamber (1) to the gas sensor (6) is created in the blood treatment device (25).
